(19) United States
(12) Patent Application Publication
(10) Pub. No.: US 2020/0051685 A1
(43) Pub. Date: Feb. 13, 2020

(54) HOLISTIC HEALTH MARKETPLACE PLATFORM

(71) Applicant: Aetna Inc., Hartford, CT (US)

(72) Inventors: Henry S. Thompson, Plantsville, CT (US); Dharmendra R. Chokshi, Farmington, CT (US); Mark Cronin, East Hampton, CT (US); Antony L. Kerz, Rocky Hill, CT (US)

(21) Appl. No.: 16/058,747

(22) Filed: Aug. 8, 2018

(51) Int. Cl. G16H 40/20; G06Q 10/10; G06Q 30/08; G16H 50/30

(52) U.S. Cl. CPC G16H 40/20; G06Q 10/109; G16H 10/60; G16H 50/30; G06Q 30/08

(57) ABSTRACT

A system configured to: (a) receive, from a computing device, service request data including a service to be performed on behalf of a member of a carrier organization; (b) receive bid data from one or more supplier devices based on the service; (c) provide the bid data to an agent device; (d) receive winning bid data from the agent device based on the bid data; (e) transmit an electronic notification to a winning supplier device associated with a winning supplier according to the winning bid data; (f) receive a first confirmation from the winning supplier device that the service is completed; (g) receive a second confirmation from a member device of the member that the service is completed; and (h) authorize payment to a financial account associated with the winning supplier based on first confirmation and second confirmation.

### BACKGROUND

Healthcare involves maintenance and improvement of an individual's health through prevention, diagnosis, and treatment of diseases, illnesses, injuries, and so on. A standardized healthcare management ecosystem involves various traditional entities providing services to the individual, be it insurance services, pharmacy services, laboratory tests, clinic and/or hospital visits, and so on. Healthcare management with these traditional entities is standardized, allowing for seamless communication between the various entities involved without much intervention from the individual. However, there are also various non-traditional entities not part of the standardized healthcare management ecosystem. These entities may be small providers that include contractors, massage therapists, yoga studios, community walking groups, and so on.

Current healthcare management technologies are not accommodating to non-traditional health related services from non-traditional providers. For example, reimbursement for a medical procedure obtained from a non-traditional provider can be a difficult process for the individual. Other deficiencies are present in healthcare management technologies as related to non-traditional health related services. For example, processes are often manual—i.e., they require individuals to locate and match suppliers to their specific needs, and require individuals to contract with suppliers with no support from their health insurance carrier. Individuals typically pay out of pocket and take a partial reimbursement risk for services rendered by the non-traditional suppliers. The claims submitted to health insurance carriers for reimbursement may or may not be covered by an individual's insurance provider.

### SUMMARY

An embodiment of the disclosure provides a system for implementing a holistic health marketplace platform. The system comprises one or more processors, which alone or in combination are configured to facilitate performing: (a) receiving, from a computing device of a carrier organization, service request data including a service to be performed on behalf of a member of the carrier organization; (b) receiving bid data from one or more supplier devices based on the service included in the service request data; (c) providing the bid data to an agent device; (d) receiving winning bid data from the agent device based on the bid data; (e) transmitting an electronic notification to a winning supplier device associated with a winning supplier according to the winning bid data; (f) receiving a first confirmation from the winning supplier device that the service is completed; (g) receiving a second confirmation from a member device of the member that the service is completed; and (h) authorizing payment to a financial account associated with the winning supplier based on first confirmation and second confirmation, wherein the winning supplier device is included in the one or more supplier devices.

An embodiment of the disclosure provides a method for providing a holistic health marketplace platform performed by a marketplace server. The method comprises: (a) receiving, from a computing device of a carrier organization, service request data including a service to be performed on behalf of a member of the carrier organization; (b) receiving bid data from one or more supplier devices based on the service included in the service request data; (c) providing the bid data to an agent device; (d) receiving winning bid data from the agent device based on the bid data; (e) transmitting an electronic notification to a winning supplier device associated with a winning supplier according to the winning bid data; (f) receiving a first confirmation from the winning supplier device that the service is completed; (g) receiving a second confirmation from a member device of the member that the service is completed; and (h) authorizing payment to a financial account associated with the winning supplier based on first confirmation and second confirmation, wherein the winning supplier device is included in the one or more supplier devices.

An embodiment of the disclosure provides an agent device associated with a marketplace agent. The agent device comprises one or more processors, which alone or in combination are configured to facilitate performing: (a) receiving, from a marketplace server, bid data based on a service included in service request data, the service being a service to be performed on behalf of a member of a carrier organization; (b) sending, to the marketplace server, winning bid data based on the bid data; and (c) receiving, from the marketplace server, a first notification that the service is completed by a winning supplier identified in the winning bid data, wherein reception of the first notification indicates that the winning supplier and the member agree that the service is completed.

### BRIEF DESCRIPTION OF THE DRAWINGS

FIG. 1 illustrates a system for a marketplace platform according to an embodiment of the disclosure;

FIG. 2 illustrates a computing device according to an embodiment of the disclosure;

FIG. 3 illustrates a system for a marketplace platform according to an embodiment of the disclosure;

FIG. 4 illustrates a process for choosing a supplier in a marketplace platform according to an embodiment of the disclosure;

FIG. 5 illustrates a communication flow using the system of FIG. 3 according to an embodiment of the disclosure;

FIG. 6 illustrates a component flow of the communication flow of FIG. 5 according to an embodiment of the disclosure;

FIG. 7 illustrates a process for registration in a marketplace platform according to an embodiment of the disclosure;

FIG. 8 illustrates a process for bid initiation in a marketplace platform according to an embodiment of the disclosure;

FIG. 9 illustrates a process for service notification in a marketplace platform according to an embodiment of the disclosure;

FIG. 10 illustrates a process for reimbursement in a marketplace platform according to an embodiment of the disclosure; and

FIG. 11 illustrates use examples of a marketplace platform according to an embodiment of the disclosure.

### DETAILED DESCRIPTION

In the current state of the art, traditional healthcare management-related technology services require pre-negotiated contracts on a national or regional level. These technology services are designed for highly standardized electronic submissions by suppliers for reimbursement. In addition, traditional healthcare management-related technology services require reimbursement to suppliers by carriers with no validation that the individual receiving the services was satisfied with the delivered services. The current state of the art is specific to traditional suppliers, e.g., providers (such as doctors or hospitals, for example), and has no mechanism to identify, inventory, credential, or contract with non-traditional suppliers, e.g., carpenter, yoga group, or community walking group, and so on. The traditional healthcare model requires that carriers pre-negotiate contracts with intermediaries to locate suppliers for a pre-determined set of services. This results in limited flexibility in services offered as well as limited cost control since these contracts are pre-negotiated and have no means of capturing member satisfaction before reimbursement is rendered.

Embodiments of the disclosure provide a holistic health marketplace platform for enabling an agent-driven marketplace for non-traditional health related services. Members (or “individuals”) can use the marketplace for their clinical and social needs within their local community. Traditional models and services require pre-negotiated contracts with pre-selected suppliers on a national or regional basis, while the agent-driven marketplace disclosed herein can accommodate agreements on a per supplier basis at a local scale. As suppliers and service variety differs by hyper-local markets, embodiments of the disclosure connect hyper-local suppliers for hyper-local community-based services with a representative of a carrier or the carrier, e.g., an insurance company, on behalf of the members of the carrier.

Advantages of the disclosed holistic health marketplace include ensuring that contracts initiated by the representative of the carrier meet the needs of the member and ensuring that the carrier is willing pay for the services (since service requests originate from the carrier). Another advantage of the disclosed holistic health marketplace includes allowing national suppliers to participate in the marketplace at a potentially lower cost.

The disclosed holistic health marketplace also removes the member's burden of identifying suppliers, reviewing contracts, and providing out of pocket payment. The disclosed holistic health marketplace can further ensure that the member is satisfied with the services delivered before any reimbursement is authorized by the carrier. In some embodiments, the disclosed holistic health marketplace can not only generate specific, personalized bids for quote, but also aggregate multiple quotes and make best fit recommendations. The disclosed holistic health marketplace provides an advantage of reducing entry barriers for suppliers of non-traditional health related services and allows the suppliers to participate and specify which services are provided within each geographic hyper-location.

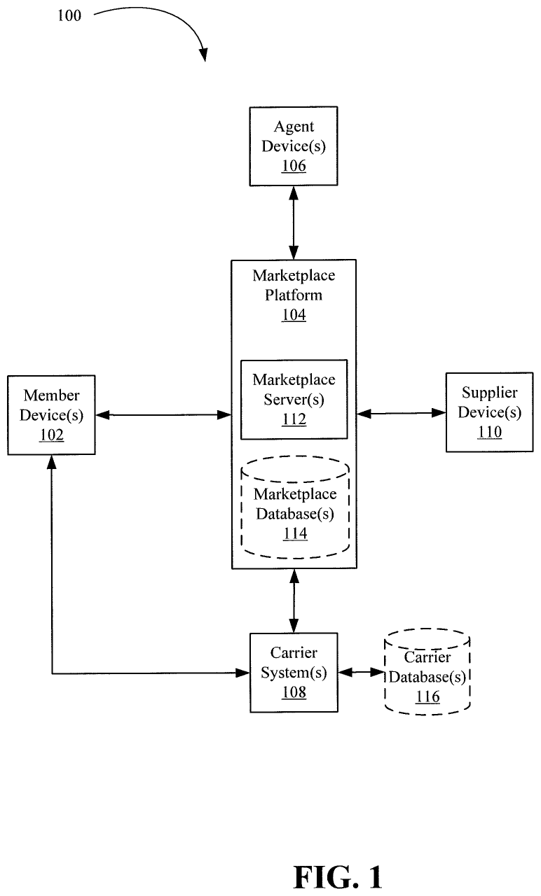

FIG. 1 illustrates a system 100 for running a marketplace platform 104 according to an embodiment of the disclosure. System 100 may include one or more member devices 102 belonging to one or more individuals seeking services, one or more agent devices 106 belonging to marketplace agents that assist in obtaining the services for the one or more individuals, one or more supplier devices 110 belonging to service suppliers, one or more carrier systems 108 and one or more carrier databases 116 for one or more carriers associated with the one or more individuals, and the marketplace platform 104. The marketplace platform 104 includes one or more marketplace servers 112 and one or more marketplace databases 114 for coordinating communication between the member devices 102, agent devices 106, carrier systems 108, and supplier devices 110. The system 100 can be used to implement an agent-assisted marketplace.

The member devices 102, the agent devices 106, and the supplier devices 110 are computing devices used by the individuals, the marketplace agents, and the suppliers, respectively. For ease of description, a single member device 102, agent device 106, and supplier device 110 are used for clarity, although it should be understood that plural member devices 102, agent devices 106, and supplier devices 110 are also within the scope of the disclosure. Examples of computing devices for the member device 102, the agent device 106, and the supplier device 110 include mobile devices, for example, a smartphone, a tablet, a phablet, a smart watch, and so on. Computing devices may also include larger devices, for example, a laptop computer, a desktop computer, and so on. Computing devices may also include communication devices for voice and/or video calls, such as, telephones and computers with microphones and cameras. Computing devices may further include personal digital assistants on smart home devices, for example, smart speakers including Amazon® Echo with Alexa digital assistant, Google® Home with Google Assistant, Apple® HomePod with Siri digital assistant, etc.

The member device 102 is named a “member” device because the individual using the device is associated with a carrier; i.e., the individual using the device is using it on behalf of a member of the carrier, or the individual is a member of the carrier. The agent device 106 is a device used by an agent associated with a marketplace to assist a member of the carrier in obtaining services using the marketplace platform 104. The supplier device 110 is a device used by a supplier to interface with the marketplace platform 104.

The carrier systems 108 are associated with one or more carriers to which a member belongs. In an example where there is only one carrier (e.g., a health insurance company), the carrier system 108 includes computing servers and devices for providing insurance services to members of the carrier. The servers in carrier system 108 facilitate the provision of, for example, managing member profiles, securing member information, providing claims management, maintaining electronic health records of members, providing web interfaces for sharing information, and so on. The carrier system 108 can rely on one or more carrier databases 116 to facilitate providing one or more services to members. In an embodiment, the carrier system 108 includes membership services, financial services, care management services, contact center services, and marketplace systems for interfacing with the marketplace platform 104. In some embodiments, the carrier system 108 includes computing devices of representatives of the carrier for requesting one or more services on behalf of a member of the carrier.

The marketplace platform 104 includes one or more marketplace servers 112 that may interface with one or more marketplace databases 114. The marketplace platform 104 receives service requests from the carrier representative via the carrier system 108, where the service requests include information about the member and the carrier that the member and/or carrier representative is associated with. Depending on the carrier, a marketplace agent familiar with the carrier is provided with the service requests by the marketplace platform 104. Using the agent device 106, the marketplace agent creates, in the marketplace platform 104, an opportunity for one or more suppliers to bid for providing the service to the member. The marketplace platform 104 provides an agent-assisted marketplace where a representative of the carrier can provide service requests via the carrier system 108, a marketplace agent via the agent device 106 and/or the marketplace platform 104 creates an opportunity for suppliers to bid on the service requests, and a supplier can bid on those requests. In some embodiments, the marketplace platform 104 may automatically create the opportunity for suppliers to bid on the service requests without assistance from the marketplace agent.

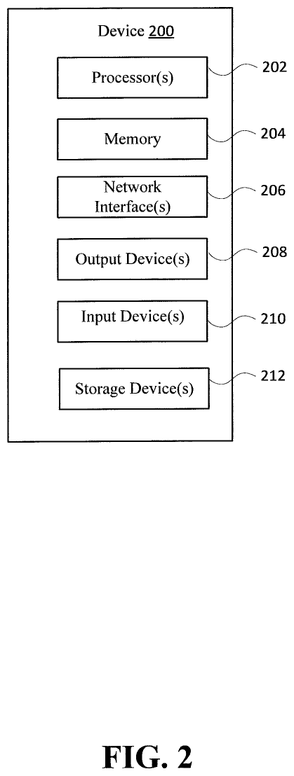

FIG. 2 is a block diagram illustrating basic hardware components of a computing device that may be used as servers, databases, member devices 102, agent devices 106, and supplier devices 110, according to some example embodiments. Device 200 may include one or more processors 202, memory 204, network interfaces 206, output devices 208, input devices 210, and storage devices 212. Although not explicitly shown in FIG. 2, each component provided is interconnected physically, communicatively, and/or operatively for inter-component communications in order to realize functionality ascribed to the member devices 102, agent devices 106, supplier devices 110, marketplace servers 112, and servers in the carrier systems 108. To simplify the discussion, the singular form will be used for all components identified in FIG. 2 when appropriate, but the use of the singular does not limit the discussion to only one of each component. For example, multiple processors may implement functionality attributed to processor 202.

Processor 202 is configured to implement functions and/or process instructions for execution within the device 200. For example, processor 202 executes instructions stored in memory 204 or instructions stored on a storage device 212. In certain embodiments, instructions stored on storage device 212 are transferred to memory 204 for execution at processor 202. Memory 204, which may be a non-transient, computer-readable storage medium, is configured to store information within the device 200 during operation. In some embodiments, memory 204 includes a temporary memory that does not retain information stored when the device 200 is turned off. Examples of such temporary memory include volatile memories such as random access memories (RAM), dynamic random access memories (DRAM), and static random access memories (SRAM). Memory 204 also maintains program instructions for execution by the processor 202 and serves as a conduit for other storage devices (internal or external) coupled to the device 200 to gain access to processor 202.

Storage device 212 includes one or more non-transient computer-readable storage media. Storage device 212 is provided to store larger amounts of information than memory 204, and in some instances, configured for long-term storage of information. In some embodiments, the storage device 212 includes non-volatile storage elements. Non-limiting examples of non-volatile storage elements include floppy discs, flash memories, magnetic hard discs, optical discs, solid state drives, or forms of electrically programmable memories (EPROM) or electrically erasable and programmable (EEPROM) memories.

Network interfaces 206 are used to communicate with external devices, computers, and/or servers. The device 200 may include multiple network interfaces 206 to facilitate communication via multiple types of networks. For example, carrier system 108 can include multiple servers connected through their network interfaces to facilitate sharing of information and making requests among the multiple servers. Network interfaces 206 may include network interface cards, such as Ethernet cards, optical transceivers, radio frequency transceivers, or any other type of device that can send and receive information. Non-limiting examples of network interfaces 206 include radios compatible with several Wi-Fi standards, 3G, 4G, Long-Term Evolution (LTE), Bluetooth®, etc.

The device 200 may also be equipped with one or more output devices 208. Output device 208 is configured to provide output to a user using tactile, audio, and/or video information. Examples of output device 208 may include a display (e.g., liquid crystal display (LCD) display, light emitting diode (LED) display, organic LED (OLED) display, microLED (mLED) display, quantum dot display, etc.), a sound card, a video graphics adapter card, speakers, magnetics, or any other type of device that may generate an output intelligible to a user of the device 200.

The device 200 may also be equipped with one or more input devices 210. Input devices 210 are configured to receive input from a user or the environment where the device 200 resides. In certain instances, input devices 210 include devices that provide interaction with the environment through tactile, audio, and/or video feedback. These may include a presence-sensitive screen or a touch-sensitive screen, a mouse, a keyboard, a camera, a microphone, a voice responsive system, or any other type of input device.

The hardware components described thus far for the device 200 are functionally and communicatively coupled to achieve certain behaviors. In some embodiments, these behaviors are controlled by software running on an operating system of the device 200.

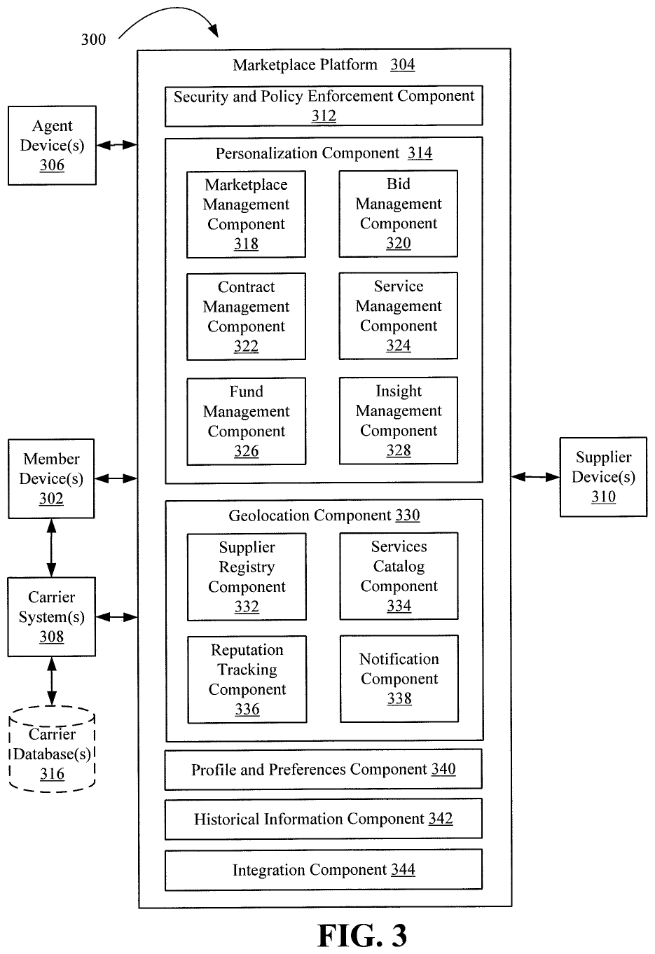

FIG. 3 illustrates a system 300 for a marketplace platform 304 according to an embodiment of the disclosure. The components of system 300 sharing the same names as those in system 100 are similar components with similar functionality, hence their description will not be repeated. That is, descriptions for member device 302, agent device 306, supplier device 310, carrier system 308, and carrier database 316 are similar to those of member device 102, agent device 106, supplier device 110, carrier system 108, and carrier database 116, respectively, and will not be repeated. Instead functional components of the marketplace platform 304 as provided in FIG. 3 will be described. The marketplace platform 304 implements one or more functional components. A functional component is a combination of hardware and software for performing one or more specific functions. For example, the security and policy enforcement component 312 includes software code executed by one or more processors of the marketplace platform 304 to implement a security protocol. The marketplace platform 304 may include the security and policy encorement component 312, the personalization component 314, the marketplace management component 318, the bid management component 320, the contract management component 322, the service management component 324, the fund management component 326, the insight management component 328, the geolocation component 330, the supplier registry component 332, the services catalog component 334, the reputation tracking component 336, the notification component 338, the profile and preferences component 340, the historical information component 342, and the integration component 344.

The personalization component 314 tailors the marketplace experience for all communications with agents, carriers, members or suppliers and works in conjunction with the marketplace management component 318 and the insight management component 328. The personalization component 314 is shown to underpin the marketplace management component 318, the bid management component 320, the contract management component 322, the service management component 324, the fund management component 326, the insight management component 328.

The marketplace management component 318 coordinates activity within the marketplace platform 304. The marketplace management component 318 coordinates the integration in and out of the marketplace platform 304, security and policy enforcement, supplier registry, services catalog, management of user accounts, and profiles and preferences. The marketplace management component 318 also handles coordination and state management to digitize end-to-end process flow for service management, bid management, contract management and fund management.

The bid management component 320 creates bids based on information from other components in the marketplace platform 304, manages bids through multiple phases, and interacts with the other components. The bid management component 320 leverages other functional components in the marketplace platform 304 to form personalized results. The bid management component 320 also creates bids, tracks bids, manages returning quotes, and so on. An advantage of the bid management component 320 is that insights, machine learning, and information gathered by other functional components in the marketplace platform 304 are brought together to create personalized bids for hyper-local areas. The personalized bids may be unique to each service request.

The contract management component 322 creates real-time contracts, where quotes are accepted from supplier devices 310. The contract management component 322 tracks and manages the various phases of a contract. These contracts are generated by obtaining information from one or more functional components. For example, a contract specifies “who, what, where, when preferences” so the contract management component 322 populates the contract by pulling specific service to be performed from the services catalog component 334, pulling supplier information from the supplier registry component 332, pulling preferences such as language from the profile and preferences component 340, and pulling location information from the geolocation component 330. In some embodiments, the contract management component 322 can be implemented in Solidity smart contracts language, leveraging blockchain technology.

The service management component 324 allows for administration of services and criteria-specific services. This is different than the services catalog component 334, which allows for registration of services as a catalog. That is, the services catalog component 334 allows for other components to identify the types of services that are available (e.g., yoga group, walking group, carpenter, etc.). This is inclusive of very specific services and credentials.

The fund management component 326 controls distribution of reimbursements to suppliers.

The insight management component 328 is inclusive of several machine learning algorithms for matching, selection, and coordination to create a personalized experience for the member, ensuring preferences and other distinguishing information is leveraged. Matching involves utilizing machine learning, insights, and other information (e.g., the service requested, the location, the preferences, the timing, etc.) to directly match needs of the member of the carrier to a specific supplier of a specific service within a specific timeframe. Coordination involves providing tracking and management of bids and quotes. Coordination provides support for information flow between components within the marketplace platform 304. Selection involves presenting a subset of quotes. Selection leverages other components in the marketplace platform 304 to ensure that the right service, in the right place, at the right time is available. Selection also takes into consideration other information (e.g., preferences, or accessibility, etc.).

The supplier registry component 332 allows suppliers to register their information with the marketplace platform 304. The supplier registry component 332 also supports the presentation of their availability to other components. This is inclusive for very specific information around geographies and availability.

The notification component 338 manages messages for multiple activities and phases. The notifications component 338 leverages information from other components as well, e.g., preference for language or medium of communication.

The reputation tracking component 336 captures information based on external and experiential information (e.g., online reviews, Better Business Bureau, member feedback, etc.), which then is codified to represent social and reputational factors of suppliers.

The profile and preferences component 340 captures supplier and carrier information relative to demographics and identification of specific preferences (e.g., carrier prefers to use veteran suppliers, supplier's preference of work locations with large vehicle access, etc.).

The historical information component 342 captures history of interactions (e.g., items such as services provided or requested, costs, specific complaints or praises, among others).

The integration component 344 provides exposure of application programming interfaces (APIs), other data integrations and user interfaces (UIs), depending on the method of accessing the marketplace platform 304.

The geolocation component 330 provides identification or estimation of the real-world geographic location of an object. The geolocation component 330 generates of a set of geographic coordinates from multiple publically available geolocation data sources. The geolocation component 330 underpins the supplier registry component 332, the services catalog component 334, the reputation tracking component 336, and the notification component 338. The geolocation component 330 is leveraged by other components in the marketplace platform 304. For example, a contract requires location information for multiple bidders in the same location. The geolocation component 330 can be used to determine services offered in specific geographies. The insight management component 328 can coordinate across multiple components, such as the profile and preferences component 340 and geolocation component 330.

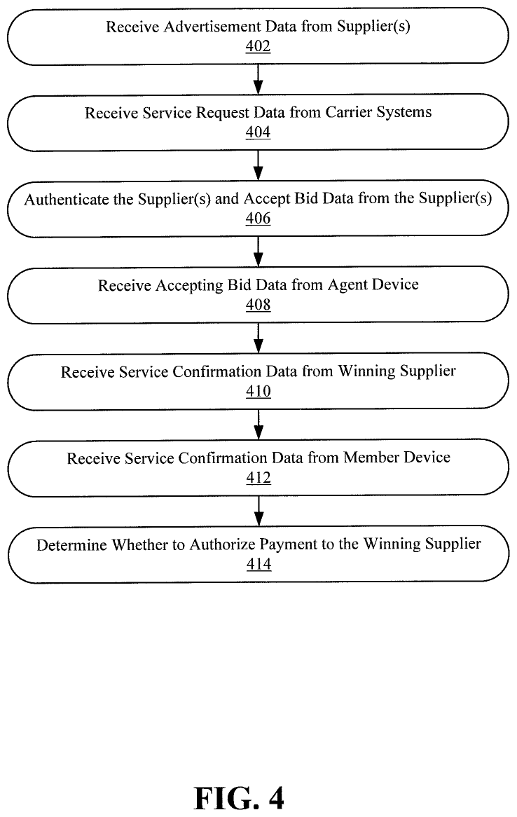

FIG. 4 illustrates a process for choosing a supplier in a marketplace platform, such as the marketplace platform 304, according to an embodiment of the disclosure. At 402, the marketplace platform 304 receives advertisement data from one or more suppliers from the supplier devices 310. The advertisement data includes services that the one or more suppliers are able to provide to members. The advertisement data can include carpentry services, community yoga services, pet therapy services, and so on.

At 404, the marketplace platform 304 receives service request data from a carrier representative. The carrier representative may provide the service request data to the marketplace platform 304 via the carrier systems 308. The service request data can include a location where a service is to be performed, whether the service to be performed requires a licensed and/or certified supplier, whether the supplier to perform the service should accommodate one or more language preferences, among other data. In an embodiment, the carrier representative determines the service request data based on information obtained from the member device 302, where the member device 302 requests a service be performed with one or more preferences. In an embodiment, the carrier representative determines the service request data based on a change in a member's personal health record or based on a conversation with the member or a reporting by the member device 302. In response to receiving the service request data, the marketplace platform 304 (e.g., with or without assistance from a marketplace agent, in various embodiments) creates a new contract record consolidating the service request data and other preferences from the carrier system 308. For example, the carrier system 308 may prefer to contract with veteran suppliers, so this is included in the created contract record. The created contract record is associated with the member, so the contract record may include a field for the responsible marketplace agent of the marketplace, a field for the member, and a field for the type of service. In an embodiment, the contract record is maintained using blockchain technology, thus the contract record is associated with an address.

At 406, the marketplace platform 304 authenticates suppliers through the supplier devices 310 and accepts bid data from the suppliers through the supplier devices 310. Suppliers matching preferences in the created contract record are informed of the contract record, and the suppliers can log in to the marketplace using supplier devices 310. The suppliers that log in can then bid to provide the service requested in the created contract record by sending bid data from the supplier devices 310 to the marketplace platform 304. The bid data may includes a price, a time estimate for completing the task, any other information that is requested in the contract record, anything else the supplier wishes to include that may be relevant, among other things.

At 408, the marketplace platform 304 receives winning bid data from the agent device 306. The bid data received from the supplier devices 310 are reviewed by the agent device 306 in conjunction with the marketplace platform 304, and the agent device 306 accepts one of the bids by providing the winning bid data to the marketplace platform 304. The marketplace platform 304 provides the winning supplier with an electronic notification informing the winning supplier of being chosen for performing/providing the requested service to the member. The created contract record is updated at this stage by adding the supplier whose bid was accepted, i.e., the winning supplier. In an embodiment, the contract record is maintained using blockchain technology, thus an account associated with the winning supplier is sent to the address associated with the contract created record at 404 along with the type of service. By sending the account associated with the winning supplier to the address associated with the contract record, the address associated with the contract record is updated to further include an identification of the winning supplier. The update can take the form of appending the identification of the winning supplier to the address. The supplier is notified of the acceptance of the bid, a contract is formed between the supplier and the marketplace platform, and the supplier proceeds to fulfill the task included in the contract.

At 410, after the task included in the contract is completed, the marketplace platform 304 and/or marketplace agent via agent device 306 receives service confirmation data from the supplier through a supplier device 310 associated with the supplier. The service confirmation data may include timing of when the service was performed by the supplier, location where the service was performed, and a request for payment authorization. In the embodiment where the contract is implemented with blockchain technology, the supplier device 310 sends a “work done” message to the address associated with the contract managed by the marketplace platform 304. In the event that the “work done” message is received properly, the marketplace platform 304 can provide an acknowledgement to the supplier device 310. The winning supplier is the only supplier that can provide the “work done” message to update the contract. The update to the contract can take the form of appending a “done” identifier to the address of the contract.

At 412, the marketplace platform 304 and/or marketplace agent via agent device 306 receives service confirmation data from the member device 302. In an embodiment, the service confirmation data received from the winning supplier at 410 is sent by the marketplace platform 304 to the member device 302, and the member device 302 confirms accuracy. In an embodiment, the service confirmation data received from the member device 302 is a service review that the member completes using the member device 302, where the service review includes a line item for indicating satisfaction with the service and for indicating that payment be made. In one embodiment where the contract is implemented with blockchain technology, the member device 302 sends a “signoff” message to the address associated with the contract managed by the marketplace platform 304. In the event that the “signoff” message is received properly, the marketplace platform 304 can provide an acknowledgement to the member device 302. The member associated with the contract is the only member that can provide the “signoff” message declaring or confirming that the service provided by the winning supplier is acceptable. The contract can be updated based on the “signoff” message by appending a “signoff” identifier to the address of the contract.

At 414, the marketplace platform 304 and/or marketplace agent via agent device 306 determines, based on the service confirmation data from the member device 302 and the service confirmation data from the winning supplier, whether to authorize payment to the winning supplier. If payment is authorized, then the marketplace platform 304 automatically initiates a deduction of funds in the carrier's financial accounts and credits the amount deducted in the winning supplier's financial accounts. When payment is authorized and confirmed, the created contract for the winning supplier is closed.

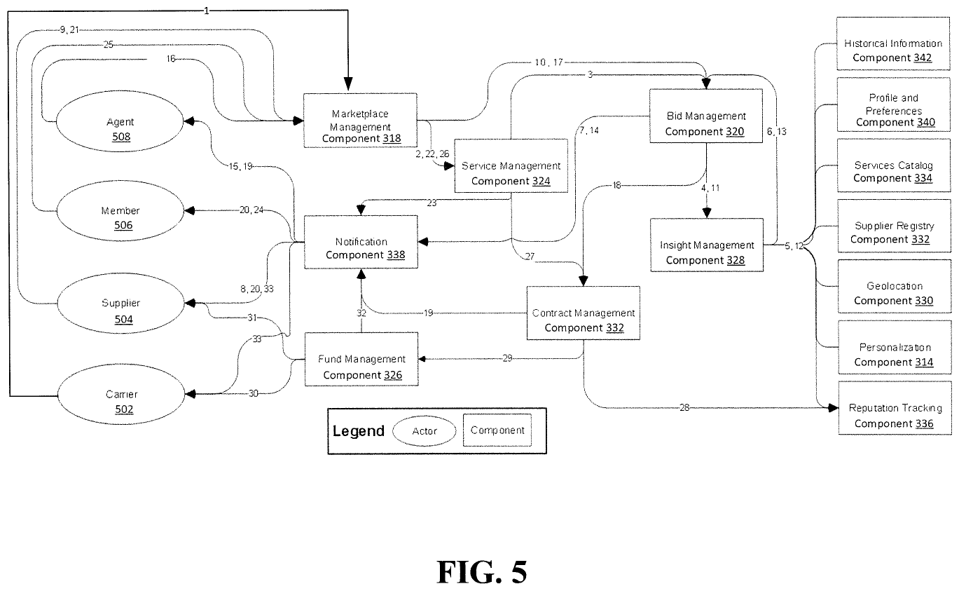

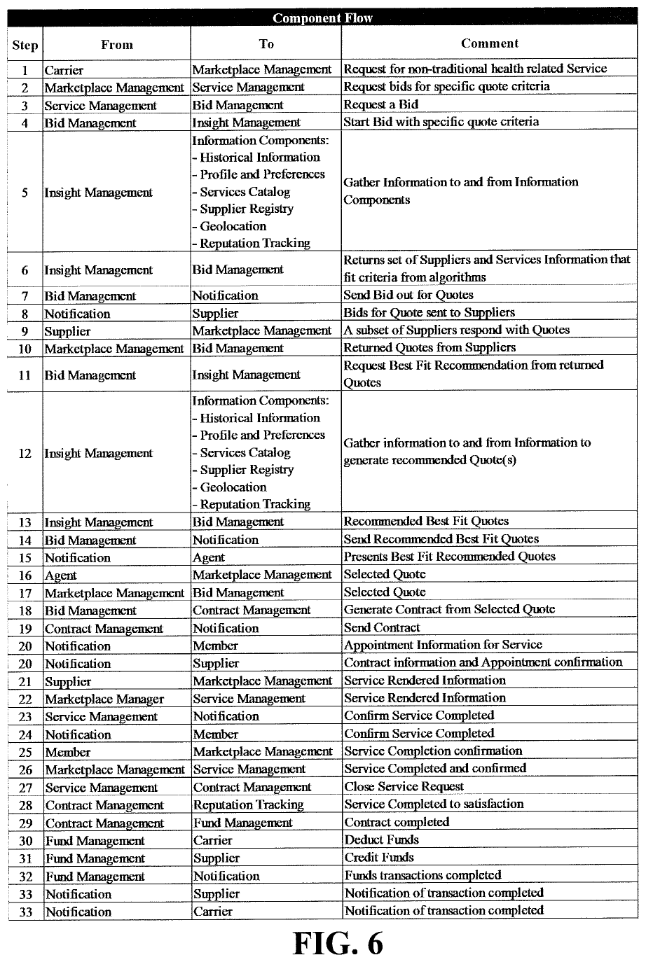

FIG. 5 illustrates a communication flow using the system 300 of FIG. 3 according to an embodiment of the disclosure, and FIG. 6 illustrates a component flow of the communication flow of FIG. 5 according to an embodiment of the disclosure. FIG. 5 shows how the different functional components communicate with one another and perform different functions to implement the process of FIG. 4. FIG. 6 indicates example information communicated at steps 1-33 of FIG. 5 and example tasks performed from one functional component to/from another functional component or to/from devices used by marketplace agents, suppliers, members, carriers, and so on. Embodiments of the disclosure provide algorithms for insight management using different kinds of information and different types of data sources. The disclosed embodiments provide a registration method and work with dynamic and non-static information sources. That is, information used by the different functional components are continually added, updated, or deleted. Using these embodiments, capturing non-traditional health related services in a hyper-localized manner can be realized. Information beyond demographics includes the services provided, the locations, the accessibility factors, the preferences and how that is algorithmically matched with the needs of the request, etc.

The marketplace management component 318 works with the other components as shown in FIG. 5. In an embodiment, once carriers 502 and suppliers 504 register, they also provide specific information on multiple criteria, e.g., a carrier 502 may indicate a preference for veteran-owned or women-owned businesses, or conversely a supplier 504 may provide language capabilities, locations of services, and information regarding their delivery of services, such as accessibility by large vehicles. The personalization component 314 use specific and unique algorithms to provide not only the general list of available services and suppliers, but also match and select best fit recommendations based on unique individual needs. Embodiments of the disclosure use ever present, continuous machine learning algorithms, updated information (including licensure, reputation, etc.) to generate recommendations that are best fit for the needs of a member 506. The functional components identified in FIGS. 5-6 have specific responsibilities, and the orchestration of the components provides the differentiated intelligence and recommendations that then facilitate an agent 508 using an agent device 306 to finalize a match.

Embodiments of the disclosure provide not only a registry of suppliers and tasks, but also information for the various parties involved. The marketplace platform 304 accounts for ensuring licensure, includes external ‘vetting’ by checking one or more databases, e.g., Dunning, Better Business Bureau, etc., and allows for non-codified services or non-traditional health services, e.g., yoga, pet therapy, walking group, and so on. The marketplace platform 304 and/or marketplace agent device 306 also allows for codification of other values and preferences, e.g., a preference for veteran suppliers, etc. The algorithms not only match, but also select and coordinate bids from suppliers. Embodiments of the disclosure also provide an automated contracting process as well as reimbursement cycles.

Traditional models and services, e.g., health insurance, require pre-negotiated contracts with pre-selected suppliers on a national or regional basis. Suppliers and service variety usually differ by hyper-local markets, therefore, embodiments of the disclosure provide a delivery method that connects hyper-local suppliers for local community-based services with members via a representative of a carrier. Embodiments of the disclosure ensure that contracts initiated by actions of the representative of the carrier will meet the needs of the member and ensures that the carrier is willing pay for the services included in the contracts. The embodiments allow national suppliers to participate in the marketplace at potentially lower costs compared to traditional models. The embodiments provide an advantage where a member's burden of identifying suppliers, reviewing contracts, and providing out of pocket payment is removed. The embodiments also ensure that the member is satisfied with the services provided before reimbursement is authorized.

Non-healthcare traditional models of online auctions and online listings focus on tangible durable and non-durable goods. These models do not include a solution for non-product items (e.g., business services). An agent-driven holistic health marketplace platform according to embodiments of the disclosure enables registration, bidding, quoting, contracting and reimbursement of services, not products.

In an embodiment, the following steps are performed in the system 300 of FIG. 3: (1) Representative of the carrier connects with Member; (2) Representative of the carrier determines Member requires non-traditional health related service; (3) Representative of the carrier requests service quote through the Holistic Health Marketplace Platform; (4) Holistic Health Marketplace Platform locates Suppliers that fit the quote criteria and distributes the quote for bids; (5) Supplier bids for service in local area through Holistic Health Marketplace Platform; (6) Holistic Health Marketplace Platform aggregates bids and creates bid recommendations for the Agent of the Marketplace; (7) Agent of the Marketplace reviews bid recommendations and accepts a bid through Holistic Health Marketplace Platform; (8) Holistic Health Marketplace Platform sends acceptance to Supplier; (9) Holistic Health Marketplace Platform notifies Member with Supplier information and appointment details; (10) Supplier renders the service to Member; (11) Supplier confirms service rendered through Holistic Health Marketplace Platform; (12) Holistic Health Marketplace Platform confirms service rendered to satisfaction with Member; (13) Holistic Health Marketplace Platform deducts funds from Carrier account in near real-time (if applicable); and (14) Holistic Health Marketplace Platform credits Supplier account in near real-time (if applicable).

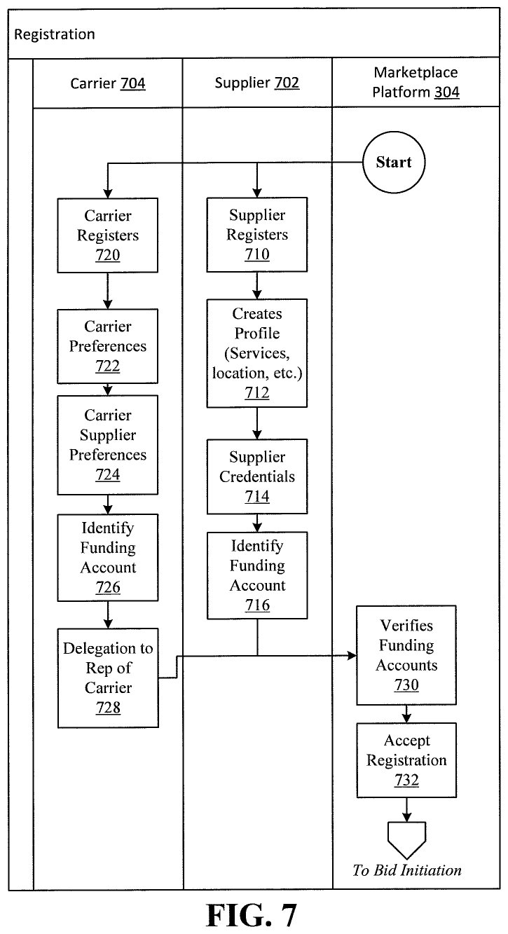

FIG. 7 illustrates a process for registration in a marketplace platform, e.g., marketplace platform 304, according to an embodiment of the disclosure. During registration, suppliers 702 and carriers 704 provide not only basic demographics. Suppliers 702 further provide their specific set of services and where the services will be provided. Supplier registration 710 involves creating a profile 712, including an expanded set of information (such as services and location) that exceeds that of a simple registry. In addition, the supplier 702 can provide credentials 714, such as licenses, and provide funding information 716. Digitization of the obtained information allows for building a registry for non-traditional suppliers 702 of any size in any location.

In FIG. 7, the marketplace platform 304 receives from the carrier 704 during carrier registration 720 carrier preferences 722, carrier supplier preferences 724, funding account information 726, and a delegation 728 of service management responsibilities to one or more carrier representatives. In FIG. 7, the marketplace platform 304 receives from the supplier device 310 a supplier profile, credentials/licenses, and funding account information. The marketplace platform 304 then verifies 730 funding accounts for each of the carrier and the supplier, and if the funding accounts are successfully verified, accepts registration 732. FIG. 7 combines the flow diagrams for the carrier 704 and the supplier 702 at the marketplace platform 304, but each path is independent of the other, for example, each verification of the funding account is performed separately, and the supplier's registration is accepted independently of the carrier's registration, and vice versa.

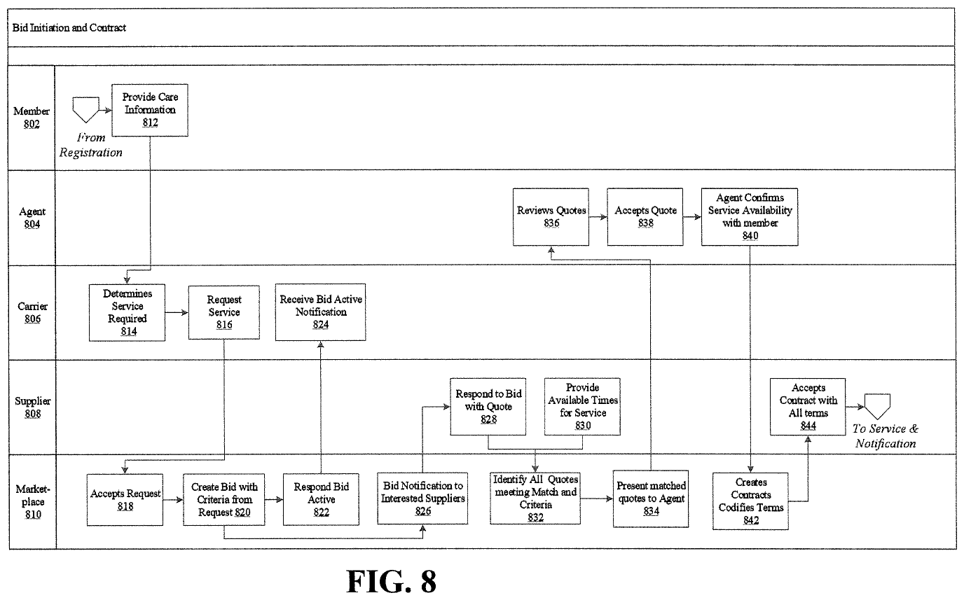

FIG. 8 illustrates a process for bid initiation in a marketplace platform 810 according to an embodiment of the disclosure. Bid initiation is an outcome of algorithms applied across the marketplace platform 810. Bid initiation involves engagement of multiple entities as indicated in FIG. 8. The bid initiation process describes how components of the system 300 interact to deliver hyper-local services meeting both a carrier's preferences and policies as well as a member's preferences and individual circumstances. Additional features of bid initiation include creation of just-in-time contracts and agreements, thus avoiding difficulty associated with pre-negotiated contracts. The just-in-time contracts and agreements allow for a widening of the supplier base and services available to a member compared to traditional pre-negotiated contracts.

In FIG. 8, member 802 registers and provides care information 812. The care information 812 could be a change in the member's personal health record (PHR). A representative of the carrier determines services 814 for the member 802 via the carrier 806 and requests a service 816 from the marketplace platform 810. The marketplace platform 810 accepts the request 818 and creates a bid 820 with criteria from the request. The marketplace platform 810 then sets the created bid as being active 822, and the carrier 806 receives a bid active notification 824. The marketplace platform 810 also provides a bid notification 826 to interested suppliers, and the supplier 808 responds to the bid with a quote 828. The supplier also provides the marketplace platform 810 with available times 830 for performing the service. The marketplace platform 810 identifies all quotes from suppliers meeting match and criteria 832. The marketplace platform 810 presents matched quotes 834 to the marketplace agent 804. The marketplace agent 804 reviews the matched quotes 836, accepts a quote 838, and confirms service availability 840 with the member 802. The marketplace platform 810 creates contracts and codifies terms 842, and the supplier 808 accepts contract with all terms 844.

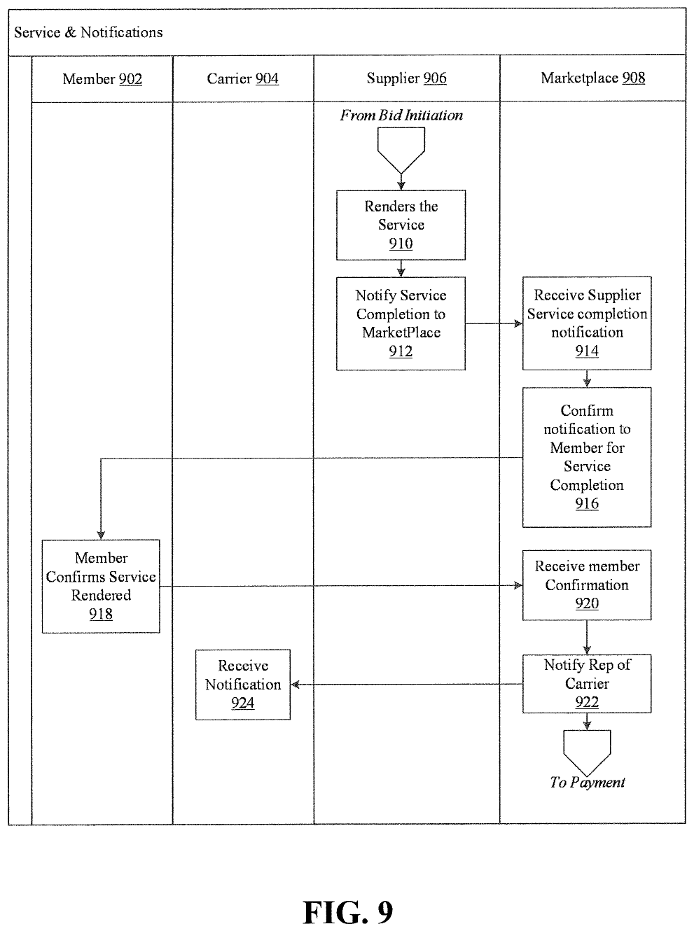

FIG. 9 illustrates a process for service notification in a marketplace platform 908 according to an embodiment of the disclosure. Service notification involves providing an outcome and ensuring that all service delivery and member interactions are managed across the marketplace platform 908. Service notification according to embodiments of the disclosure describes a disruptive approach to ensure that a member is no longer searching, contracting and scheduling services with no support. Rather it describes how the components of the system 300 provide that outcome and digitize many processes.

Supplier 906 renders the service 910 and notifies the marketplace platform 908 of service completion 912. The marketplace platform 908 receives supplier service completion notification 914. The marketplace platform 908 confirms notification to member 902 for service completion 916. The member 902 confirms service rendered 918. The marketplace platform 908 receives member confirmation 920 and then sends a notification 922 to a representative of the carrier 904. The marketplace platform 908 may also send the notification to the marketplace agent (i.e., to the agent device). The representative of the carrier 904 receives notification 924.

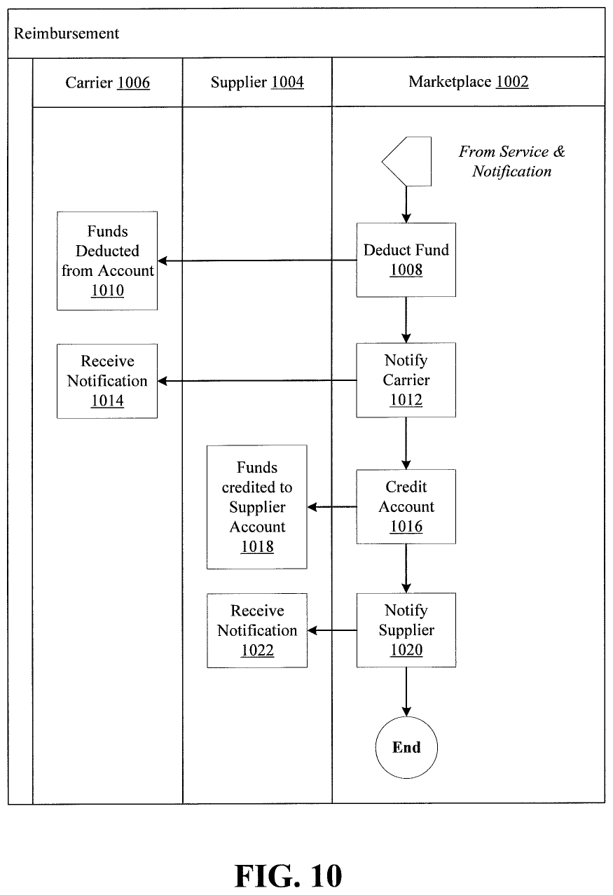

FIG. 10 illustrates a process for reimbursement in a marketplace platform 1002 according to an embodiment of the disclosure. Due to the registration of suppliers 1004 and carriers 1006 with the marketplace platform 1002, reimbursement according to embodiments of the disclosure ensure that the member is not caught in-between the carrier and the supplier. Rather, just-in-time contracting, enabled by the intelligence derived from algorithms from highly personalized information for hyper localized service delivery, will be used for direct reimbursement in near real-time.

Marketplace platform 1002 deducts funds 1008 which triggers funds to be deducted from account 1010 of the carrier 1006. The marketplace platform 1002 sends a notification 1012 to the carrier 1006, and the carrier 1006 receives notification 1014. The marketplace platform 1002 credits account 1016 of the supplier 1004 which triggers funds to be credited to account 1018 of the supplier 1004. The marketplace platform 1002 then sends a notification 1020 to the supplier 1004, and the supplier 1004 receives notification 1022.

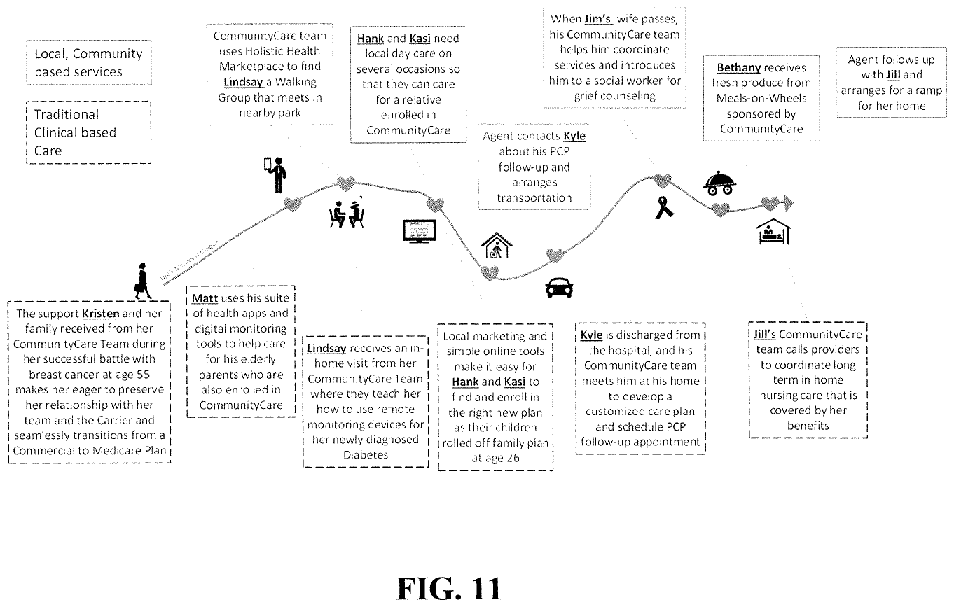

FIG. 11 illustrates use examples of a marketplace platform according to an embodiment of the disclosure. Examples of traditional clinical-based care are illustrated alongside examples of local, community-based services that are arranged through the marketplace platform. The local, community based services include, e.g., walking groups, daycare, transportation, grief counseling provided by a social worker, obtaining meals and other services from charities, construction of an accessibility ramp, etc.

Embodiments of the disclosure provide an infrastructure or platform to create an agent assisted marketplace for connecting buyers and suppliers. Any healthcare company that wants to provide non-traditional health related services for their patients or members can engage in the marketplace. These healthcare companies or entities may include state or federal governments, carriers, health systems, pharmacy benefit management (PBM) entities, hospitals, and so on.

Embodiments of the disclosure improve upon state of the art in that the state of the art has no mechanism to locate and purchase non-traditional health related services that vary by local market. These services are part of the member care model, so by including them in a marketplace, embodiments of the disclosure address social determinants that drive the vast majority of medical cost. Unlike the state of the art, a holistic health marketplace platform according to embodiments of the disclosure will digitize multi-step processes across multiple stakeholders involved in securing and delivering the services. As a result, embodiments of the disclosure: (a) ensure services are completed to the satisfaction of the member before reimbursement is rendered; (b) enable low cost direct commercial relationships without intermediaries; and (c) mitigate risk of sourcing new suppliers within a local geography by allowing hyper-local suppliers to provide services at potentially lower cost on an as needed basis.

Embodiments of the disclosure provide a holistic health marketplace platform that implements an agent driven marketplace for non-traditional health related services. Suppliers of the services meet locally with a member or vice versa for the member's clinical and social needs within the community. Traditional models and services require pre-negotiated contracts with pre-selected supplier on a national or regional basis. As suppliers and service variety will differ by local market, the marketplace connects hyper-local suppliers for local community based services with the agent of a carrier on behalf of members. Complementary to the state of the art, the marketplace is open to national suppliers, but the national suppliers can provide their services at a potentially lower cost.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A system for implementing a health marketplace platform, the system comprising one or more processors, which alone or in combination are configured to facilitate performing:
receiving, from a computing device of a carrier organization, service request data including a service to be performed on behalf of a member of the carrier organization;
receiving bid data from one or more supplier devices based on the service included in the service request data;
providing the bid data to an agent device;
receiving winning bid data from the agent device based on the bid data;
transmitting an electronic notification to a winning supplier device associated with a winning supplier according to the winning bid data;
receiving a first confirmation from the winning supplier device that the service is completed;
receiving a second confirmation from a member device of the member that the service is completed; and
authorizing payment to a financial account associated with the winning supplier based on first confirmation and second confirmation,
wherein the winning supplier device is included in the one or more supplier devices.

2. The system according to claim 1, wherein the one or more processors are further configured to facilitate performing:
receiving advertisement data from the one or more supplier devices;
wherein the advertisement data matches the type of service in the service request data, and wherein the type of service includes carpentry services, community yoga services, or pet therapy services.

3. The system according to claim 1, wherein the service request data further comprises one or more of:
a location where the service is to be performed;
a licensing requirement for the service to be performed; and
a language preference for a supplier of the service.

4. The system according to claim 1, wherein the one or more processors are further configured to facilitate performing:
creating a contract between the winning supplier and the carrier organization for the benefit of the member based on the service request data.

5. The system according to claim 4, wherein the contract is further created based on preferences of the carrier organization or preferences of the member.

6. The system according to claim 4, wherein the one or more processors are further configured to facilitate performing:
updating the contract to identify the winning supplier based on the winning bid data.

7. The system according to claim 4, wherein the one or more processors are further configured to facilitate performing:
updating the contract based on the first confirmation from the winning supplier device; and
updating the contract based on the second confirmation received from the member device.

8. The system according to claim 1, wherein the one or more processors are further configured to facilitate performing:
creating a contract between the winning supplier and the carrier organization for the benefit of the member, wherein the contract is referenced by an address using blockchain technology.

9. The system according to claim 8, wherein the one or more processors are further configured to facilitate performing:
updating the contract to identify the winning supplier based on the winning bid data by appending identifying information of the winning supplier to the address.

10. The system according to claim 8, wherein the one or more processors are further configured to facilitate performing:
updating the contract based on the first confirmation received from the winning supplier device by appending an identifier that indicates the winning supplier has completed the service included in the service request data; and
updating the contract based on the second confirmation received from the member device by appending an identifier that indicates the service completion is acceptable to the member.

11. A method for providing a health marketplace platform performed by a marketplace server, the method comprising:
receiving, from a computing device of a carrier organization, service request data including a service to be performed on behalf of a member of the carrier organization;
receiving bid data from one or more supplier devices based on the service included in the service request data;
providing the bid data to an agent device;
receiving winning bid data from the agent device based on the bid data;
transmitting an electronic notification to a winning supplier device associated with a winning supplier according to the winning bid data;
receiving a first confirmation from the winning supplier device that the service is completed;
receiving a second confirmation from a member device of the member that the service is completed; and
authorizing payment to a financial account associated with the winning supplier based on first confirmation and second confirmation,
wherein the winning supplier device is included in the one or more supplier devices.

12. The method according to claim 11, further comprising:
receiving advertisement data from the one or more supplier devices;
wherein the advertisement data matches the type of service in the service request data, and wherein the type of service includes carpentry services, community yoga services, or pet therapy services.

13. The method according to claim 11, wherein the service request data further comprises one or more of:
a location where the service is to be performed;
a licensing requirement for the service to be performed; and
a language preference for a supplier of the service.

14. The system according to claim 11, further comprising:
creating a contract between the winning supplier and the carrier organization for the benefit of the member based on the service request data.

15. The method according to claim 14, wherein the contract is further created based on preferences of the carrier organization or preferences of the member.

16. The method according to claim 14, further comprising:
updating the contract to identify the winning supplier based on the winning bid data.

17. The method according to claim 14, further comprising:
updating the contract based on the first confirmation from the winning supplier device; and
updating the contract based on the second confirmation received from the member device.

18. The method according to claim 11, further comprising:
creating a contract between the winning supplier and the carrier organization for the benefit of the member, wherein the contract is referenced by an address using blockchain technology.

19. An agent device associated with a marketplace agent, the agent device comprising one or more processors, which alone or in combination are configured to facilitate performing:
receiving, from a marketplace server, bid data based on a service included in service request data, the service being a service to be performed on behalf of a member of a carrier organization;
sending, to the marketplace server, winning bid data based on the bid data; and
receiving, from the marketplace server, a first notification that the service is completed by a winning supplier identified in the winning bid data, wherein reception of the first notification indicates that the winning supplier and the member agree that the service is completed.

20. The agent device according to claim 19, wherein the service request data is determined from care information provided by a member device.
